(19) United States
(12) Patent Application Publication
(10) Pub. No.: US 2010/0255468 A1
(43) Pub. Date: Oct. 7, 2010

(54) METHOD OF ASSESSING GENE EXAMINATION DATA, PROGRAM THEREFOR AND APPARATUS OF THE SAME

(76) Inventors: Kohshi Maeda, Tokai (JP); Shinichi Fukuzono, Hitachinaka (JP); Koichi Sugano, Urayasu (JP)

Correspondence Address: MCDERMOTT WILL & EMERY LLP, 600 13TH STREET, N.W., WASHINGTON, DC, 20005-3096, US

(21) Appl. No.: 12/524,681

(22) PCT Filed: Jan. 10, 2008
(86) PCT No.: PCT/JP2008/050166

§ 371 Date: Jul. 27, 2009

(30) Foreign Application Priority Data
Jan. 31, 2007 (JP) 2007-021407

(51) Int. Cl. C12Q 1/68; C12M 1/34

(52) U.S. Cl. 435/6; 435/287.2

(57) ABSTRACT

The present invention provides a means of determining the presence or absence of chromosomal abnormalities with high reliability and high specificity in a gene examination in which a test sample is compared with a standard sample in terms of changes in quantitative ratios of alleles (particularly when test results are close to the threshold).

In the present invention, alleles each of which exhibits quantitative changes when compared with those in a standard sample are identified based on quantitative changes in alleles in two or more and preferably three or more linked polymorphism sites. Then, the presence or absence of chromosomal abnormalities is determined based on the occurrence frequency of a combination of the alleles when the allele combination is considered as a haplotype.

### TECHNICAL FIELD

The present invention relates to a method of determining the presence or absence of chromosomal abnormalities by examining changes in quantitative ratios of alleles in a standard sample and in test samples. It also relates to a gene examination apparatus (system) and the like using the method.

### BACKGROUND ART

In a method of examining chromosomal abnormalities by comparing a test sample with a standard sample in terms of changes in quantitative ratios of alleles, the accuracy of the examination highly depends on whether the presence or absence of such changes can be accurately determined. For instance, in the case of LOH assay in which examined regions in nucleic acids in a test sample are amplified by a PCR-SSCP method and quantitative ratios of amplified alleles are examined, the presence or absence of cancer cells in the test sample is determined based on changes in quantitative ratios of alleles. In this case, the assay reliability is significantly influenced by how chromosomal abnormalities are determined based on the detected changes in the quantitative ratios of the alleles in the test sample. Particularly in the case of a gene examination for a bladder cancer, in which nucleic acids in cells collected from urine are examined in terms of LOH, it is highly probable that urine contains normal cells in addition to cancer-derived cells floating therein. Therefore, a high levels of determination accuracy is required. In the case of nucleic acid sequencing analysis using a DNA sequencer, it is also possible to examine quantitative ratios of alleles concomitantly with detecting the nucleic acid sequence and polymorphisms (JP Patent Publication (Kohyo) No. 2006-508632 A). However, in the case of this analysis method, it is also important to determine whether changes in the quantitative ratios of alleles are nonsignificant changes caused by measurement variation or significant changes representing chromosomal abnormalities.

As can be seen from the above examples, when determining whether there are changes in the quantitative ratios of alleles in the test sample by comparing with a standard sample, it is necessary to use some sort of indicators in order to determine that changes in the quantitative ratios of the alleles are significant. A conventional method of determining quantitative ratios of alleles has been conducted as follows. The quantitative ratio of alleles is measured more than once using an identical method with the use of a standard sample. Then, a measurement variation of the quantitative ratio of the alleles is examined. If the test results for test samples indicate changes that are more statistically significant than measurement variation, the results are designated as “positive” (JP Patent Publication (Kokai) No. 2001-112499 A; US2003/0082616 A1). It has been very difficult to determine the threshold for determination of whether the changes in the quantitative ratio of the alleles are significant. This is because, if the range of negative results is set wide, false negatives increase, while, if the range of positive results is set wide, false positives increase. In addition, if results close to the threshold are determined to fall within the range of undetermined results, results impossible to determine increase.

Further, it has been recently found that when the quantity of genomic nucleic acids used for measurement is small, the degree of measurement variation in terms of the quantitative ratios of the alleles is large. Accordingly, it has been found that when a small quantity of genomic nucleic acid is used for the examination, the reliability of determination using a threshold that is determined by the conventional method decrease. Therefore, a new invention has been suggested, whereby a highly reliable threshold can be calculated based on the quantity of a genomic nucleic acid to be amplified (JP Patent Publication (Kokai) No. 2006-87388 A).

### DISCLOSURE OF THE INVENTION

### Problem to be Solved by the Invention

In relation to a threshold for determination in gene examination in which the presence or absence of chromosomal abnormalities is determined by comparing changes in quantitative rations of alleles in a test sample with a standard sample, if the range of negative results is set wide, false negatives increase, while, if the range of positive results is set wide, false positives increase. In addition, if results close to the threshold are determined to fall within the range of undetermined results, results impossible to determine increase. In particular, when test results are close to the threshold, it has been difficult to assess such test results with high reliability and high specificity.

### Means for Solving Problem

In order to determine the presence or absence of a small quantity of cancer cells with high accuracy, the present inventors have conducted a variety of studies during the research process for gene examination in which cancer cells in urine are examined in terms of quantitative ratios of alleles in a plurality of polymorphism sites. As a result, the present inventors have discovered that there are significant deviations in the frequencies of a plurality of possible allele combinations, and that false positives would be obtained with higher possibility in the cases of low-frequency allele combinations.

Specifically, the present invention provides, as a means for solving the above problems, a method of determining chromosomal abnormalities in a test sample by examining quantitative ratios of alleles in genetic polymorphism sites, comprising the steps of: measuring quantitative changes in alleles in two or more linked polymorphism sites to identify alleles each of which exhibits quantitative changes when compared with those in a standard sample; and determining the presence or absence of chromosomal abnormalities based on the occurrence frequency of a combination of the alleles, given that the allele combination is considered as a haplotype. In the present invention, chromosomal abnormalities include DNA structural abnormalities and copy number variations.

For instance, the method of the present invention can be carried out by the following steps:

1) calculating a reliability of quantitative changes in alleles exhibiting changes in gene quantity when compared with those in a standard sample;

2) determining the presence or absence of chromosomal abnormalities based on the quantitative changes in the alleles and the occurrence frequency of a combination of the alleles; and

3) comparing the occurrence frequency of the combination of the alleles and the reliability of the quantitative changes in the alleles to designate the higher result as a final determination result.

As rough guidelines for determination, the test sample is determined as having chromosomal abnormalities when the occurrence frequency of the above combination of the alleles is 1% or more, preferably 5% or more, and more preferably 20% or more, given that the allele combination is considered as a haplotype.

Specifically, quantitative ratios of alleles are measured using, as examined regions, two or more linked polymorphism sites and more preferably three or more linked polymorphism sites in a test sample. Further, a combination of alleles which have relatively decreased in quantity compared with corresponding alleles, or a combination of alleles showing no quantitative decrease is examined. The quantitative ratios of the alleles in a standard sample (i.e., standard data) are compared with the quantitative ratios of the alleles in the test sample and then the obtained test result is subjected to primary determination based on the predetermined threshold for determination. In a case that the test result subjected to primary determination is found to be “positive,” the occurrence frequency of the combination of the alleles is examined. If the occurrence frequency is high, the result is designated as “positive.” If it is low, the result is designated as “negative.” Even when the test result subjected to primary determination is designated as “negative,” it can be re-designated as “positive” if the allele combination obtained from the test result is that with high occurrence frequency, while the test result can be re-designated as “negative” if the allele combination is that with low occurrence frequency.

The method of the present invention is useful especially for examining a test sample that is difficult to determine in terms of chromosomal abnormalities based on quantitative changes in alleles since in the test sample the changes in quantity of the gene are close to the predetermined threshold for determination; that is, changes fall within an error range. For instance, the range of low-reliability results for which “the changes in quantity of the gene are within an error range” is considered as undetermined results. For a test sample the test result from which is considered as undetermined, if the allele combination obtained from the test result is that with high occurrence frequency, the test result is designated as “positive,” while, if the combination is that with low occurrence frequency, the test result is designated as “negative.”

In the present invention, the determination of chromosomal abnormalities may be carried out with the use only of the occurrence frequency or the occurrence probability as an indicator for determination without primary determination based on quantitative changes in alleles. Specifically, particular polymorphisms are examined as examined regions to measure allele quantities. A combination of two alleles, one of which has relatively decreased in quantity, or a combination of alleles showing no quantitative decrease is examined. Subsequently, the combination of alleles from the test result is compared with a combination of alleles with high occurrence frequency. If the combination of alleles from the test result corresponds to a haplotype with high occurrence frequency, the result is designated as “positive.” If not, the result is designated as “negative.”

According to the present invention, a program or an apparatus (system) for determining chromosomal abnormalities by examining quantitative ratios of alleles in genetic polymorphism sites is also provided. Such program or apparatus (system) comprises: a means of inputting (measuring) quantitative changes in alleles in two or more, and more preferably three or more, linked polymorphism sites in a test sample for comparison with a standard sample; a means of identifying alleles each of which exhibits quantitative changes when compared with those in a standard sample; and a means of determining the presence or absence of chromosomal abnormalities based on the occurrence frequency of a combination of the alleles, given that the allele combination is considered as a haplotype

The program and the apparatus of the present invention may further comprise the following means:

1) a means of calculating a reliability of quantitative changes in alleles exhibiting quantitative changes compared with those in a standard sample;

2) a means of determining the presence or absence of chromosomal abnormalities based on the quantitative changes in the alleles and the occurrence frequency of a combination of the alleles; and

3) a means of comparing the occurrence frequency of the combination of the alleles and the reliability of the quantitative changes in the alleles, determining the higher result as a final determination result, and outputting the result.

In addition, the program or the apparatus of the present invention may comprise a means of outputting (presenting) the reliability of final determination results or a means of outputting (presenting) the combination of alleles, the occurrence frequency thereof, the reliability of quantitative changes in the alleles, and the reliability of final determination results.

Further, the program or the apparatus of the present invention may comprise a means of storing various forms of data from a test sample obtained by examination, including the occurrence frequencies of the combinations, and the occurrence frequency of various haplotypes obtained by re-calculation with the addition of the data.

Specifically, the program or the apparatus of the present invention has a function of capturing measurement data from detectors or apparatuses for measuring the quantitative ratios of alleles in standard samples or test samples. In addition, it comprises: a database of standard data of the quantitative ratios of alleles at various polymorphism sites in healthy individuals; a database of allele combinations at polymorphism sites to be examined and the occurrence frequencies thereof; and a database of the reliability of statistically calculated quantitative changes and the threshold by which the quantitative changes are determined as significant based on the reliability. Further, it desirably comprises the following functions: a function of determining a polymorphism type in each polymorphism region based on the comparison of a standard sample or standard data and measured data from a test sample; a function of determining a combination of alleles in a test sample; a function of calculating quantitative allelic changes in a test sample based on the comparison of a standard sample or standard data and measured data from a test sample; a function of carrying out primary determination (positive/negative/undetermined) for quantitative allelic changes in various polymorphism sites with the use of a database for the reliability of quantitative changes and the threshold for determination; a function of comparing allele combinations in a test sample with a database for allele combinations in polymorphism sites to be examined and the occurrence frequencies thereof to store the occurrence frequencies of the allele combinations in the test sample, and to display the occurrence frequencies on a screen; a function of assigning “positive/negative” designation based on the occurrence frequencies; and a function of calculating a reliability of the determination results and presenting the results.

### EFFECTS OF THE INVENTION

According to the present invention, it is enabled to reduce the probability of erroneous designations such as “false positive” or “false negative” designations when determining the presence or absence of changes in the quantitative ratio of alleles in a test sample by comparing the test sample with a standard sample.

### BRIEF DESCRIPTION OF THE DRAWINGS

FIG. 1 shows a flowchart of the LOH determination method used in the Examples of the present invention.

FIG. 2 shows a construction of an apparatus of the present invention for detecting quantitative changes in chromosomes.

FIG. 3 shows an example of a display screen of the present invention upon a final determination.

FIG. 4 shows an example of a display screen upon the final determination in Example 1.

FIG. 5 shows an example of a display screen upon the final determination in Example 1 in which two of five alleles are homozygous.

### EXPLANATION OF REFERENCE NUMERALS

1: Detecting the allele quantity in a test sample; 2: Comparing function; 3: Function of determining polymorphism combinations; 4: First determination function; 5: Second determination function; 6: Final determination function; 7: Function of calculating and displaying reliabilities of determination results; 10: Detector or detection apparatus; 11: Database 1 of standard data of the quantitative ratios of alleles in various polymorphisms in healthy individuals; 12: Database 2 for the thresholds of various test markers; 13: Database 3 for polymorphism combinations to be examined and the occurrence frequencies thereof; 14: Comparing function; 15: Function of determining polymorphism combinations; 16: First determination function; 17: Second determination function; 18: Final determination function; 19: Memory device; 20: Output apparatus.

This specification includes the contents disclosed in the specification and drawings of Japanese Patent Application No. 2007-21407, which is a priority document of the present application.

### BEST MODE FOR CARRYING OUT THE INVENTION

Hereinafter, the procedures used in the present invention will be described with reference to the drawings. However, the scope of the present invention is not limited to the examples described below.

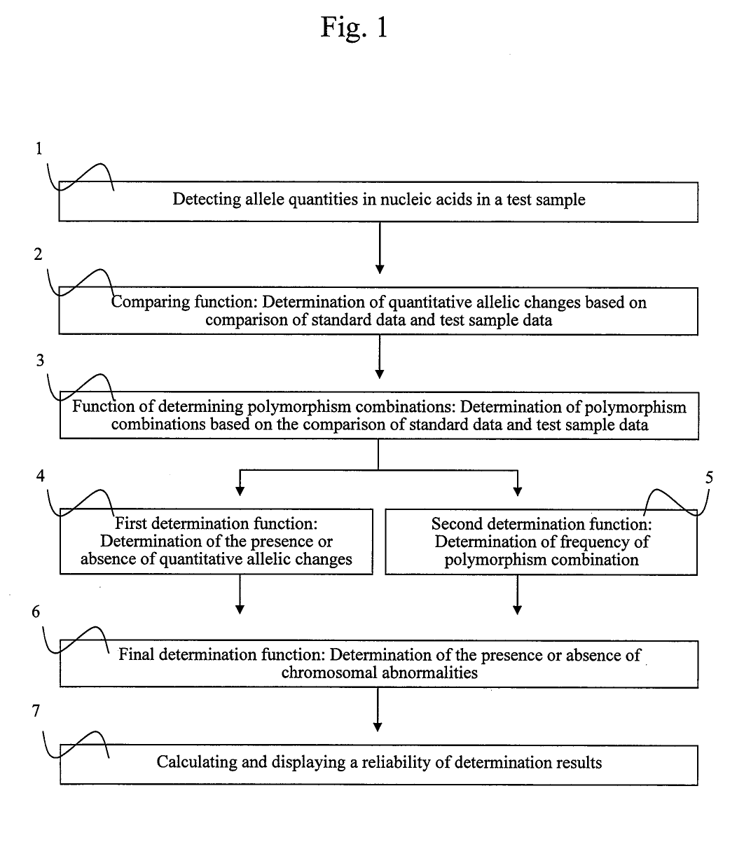

FIG. 1 shows a flowchart of the LOH determination method used in the Example of the present invention. First, a test sample prepared by extracting nucleic acids from a biological sample is subjected to detection of the allele quantity and the obtained data are captured.

In an examination utilizing the present invention, the quantitative ratios of alleles in genetic polymorphism sites are examined to determine the presence or absence of chromosomal abnormalities (chromosome doubling or deletion) in a test sample. As described above, chromosomal abnormalities include DNA structural abnormalities and copy number variations. Specifically, examples of the above test include LOH assay for detection of loss of heterozygosity (hereafter abbreviated as LOH), a CGH (Comparative Genomic Hybridization) test for detecting chromosome doubling or deletion, and sequence analysis for gene sequence determination with measurement of the quantitative ratios of alleles. The term “copy number variations” refers to variations in a copy number of genes between individuals as a result of increases in or deletion of 1-kb or larger DNA regions. The present invention is a method enabling to determine changes in copy numbers of genes in linked chromosomal regions more accurately than before. Of course, the present invention is also effective for detection of copy number variations. More specifically, two or more linked single nucleotide polymorphism sites in a region suspicious to contain copy number variations are separately amplified and alleles exhibiting changes in each single nucleotide polymorphism site are detected. In a case in which the occurrence frequency of the detected diplotype comprising the alleles is high, it is possible to determine that copy number variations are present as a result of increases in or deletion of DNA regions.

A “test sample” used in the present invention is a sample containing nucleic acids extracted from a biological sample. Examples thereof include: samples collected upon medical check such as mass examination, physical examination, complete medical examination, or physical examination using mailable kits; biological samples containing nucleic acids from human such as blood, tissue, urine, and the like collected from outpatients and inpatients at hospitals; and samples containing nucleic acids extracted from substances adhered by the above biological samples. In addition, nucleic acids to be examined are those containing linked polymorphisms. Nucleic acids can be extracted from biological samples by any known methods such as the phenol-chloroform method (a method comprising separating nucleic acids from protein components and extracting nucleic acids with phenol/chloroform) or a method comprising allowing nucleic acids to adsorb to a silica column, washing the column, and eluting the nucleic acids with a nucleic-acid-eluting solution.

The term “allele” used in the present invention refers to a difference observed at an identical locus, such difference being derived from a difference between DNA nucleotide sequences. Herein, an uppercase alphabetical letter such as “A” and a lowercase alphabetical letter such as “a” are used as symbols representing alleles. Thus, when alleles in examined polymorphism sites are homozygous, they are expressed by “AA” or “aa.” When they are heterozygous, they are expressed by “Aa.”

Next, a comparing function is used to compare measured data for a test sample with concomitantly measured data for a standard sample or with standard data stored in the database 1 to calculate quantitative allelic changes in a test sample. The term “standard sample” used herein refers to a sample that can be used as a control sample for a test sample. Specifically, when a test sample contains nucleic acids extracted from a cancer tissue collected from a bladder of a bladder cancer patient, a sample containing nucleic acids extracted from blood of the patient can be used as a standard sample. The term “standard data” refers to data obtained by detecting the allele quantities or the quantitative ratios of alleles in a standard sample that has been preliminarily measured under the same conditions as those used for measurement of the allele quantities of a test sample. A database containing such data is stored in an apparatus used in the present invention.

Subsequently, a function of determining allele combinations is used to determine a combination of linked alleles in the test sample. The allele combination can be determined by examining a combination of alleles which have relatively decreased in quantity compared with the corresponding alleles, or a combination of alleles showing no quantitative decrease. When the examined polymorphism is homozygous, the polymorphism is designated as a homozygous polymorphism. The term “an allele combination” or “a combination of alleles” in the present invention refers to a combination of at least two different alleles existing on a single chromosome. A single test sample contains two chromosomes, which are paternal and maternal chromosomes. Thus, when two polymorphism sites in a test sample are examined, two different allele combinations, one being a paternal allele combination and the other being a maternal allele combination, are simultaneously detected. Therefore, if both paternal and maternal allele combinations result in an identical polymorphism, a combination of homozygous alleles is detected. If different polymorphisms are observed, a combination of heterozygous alleles is detected. An allele combination is determined by separately detecting at least two linked polymorphism sites. The type and the number of polymorphisms and methods for detecting polymorphism sites are not particularly limited. More preferably, in order to improve a determination reliability, it is better to use a combination of alleles at three or more linked single nucleotide polymorphisms.

Next, a first determination function is used to compare allele combinations observed in the test sample with allele combinations contained in a database of occurrence frequencies of all combinations of alleles to be examined. When a high occurrence frequency or probability is obtained for the allele combination observed in the test sample, the result is designated as “positive.” When it is low, the result is designated as “negative.” Then, a reliability of the combination is calculated.

The term “an occurrence frequency” used in the present invention refers to the occurrence frequency of a combination of a plurality of alleles in genomic regions in a particular population. When polymorphisms on alleles are considered as a haplotype, it is thought that there are deviations in the occurrence frequencies of individual combinations. The statistical occurrence frequency for each combination can be calculated based on a database containing haplotype analysis results. Alternatively, it can be calculated based on a polymorphism analysis of approximately 100 individuals. In addition, it is also possible to achieve a more accurate occurrence probability by storing the allele combination occurrence frequency information in a database during actual examinations and continuously updating the information. However, according to the present invention, the method for calculating the allele combination occurrence frequency is not limited to the above examples as long as the frequency can be obtained. The occurrence frequency would vary depending on polymorphisms to be detected and purposes of the examination. However, when the occurrence frequency is generally 1% or higher, preferably 5% or higher, and more preferably 20% or higher, the result can be designated as “positive.” When it is below the above range, the result can be designated as “negative.”

The expression “a reliability of a combination” used in the present invention refers to the occurrence probability obtained when the combination of two is chosen from all possible combinations of alleles to be examined. More precisely, the term includes the probability that such allele combinations are accidentally chosen. More specifically, even if an occurrence probability of a certain combination of two alleles is 100%, the probability cannot be indeed 100% since other combinations of said two alleles may be observed due to measurement variation in a test sample even if there is no change in allele quantities. In general, a cutoff value is determined considering measurement variation. Thus, it is thought that there is very little probability that false positives would be observed by exceeding the cutoff value. However, when it is presumed that measurement variation increases for some reasons, it is not absolutely necessary that “a reliability of a combination=an occurrence probability.” It is possible to add an accidentally occurring error due to a distribution of variation to the above relationship. Of course, the relationship “a reliability of a combination=an occurrence probability” is applicable to a case that such an error is very small. In addition, although the reliability of a positive result is explained above, it is also possible to calculate the reliability of a negative result in an opposite manner. More specifically, if the determination is negative when the occurrence probability of a detected combination of alleles at 5%, the reliability of the negative result is 95%. The above calculation of the reliability of a negative result is an example in which an error due to measurement variation is not considered. The present invention is not limited to the above example.

The term “an occurrence probability” used in the present invention is more specifically described. The term refers to a probability of occurrence of a specific diplotype. In other words, the term refers to a probability obtained when a combination of two is chosen from particular combinations of a plurality of alleles to be examined. Herein, the term “particular combinations” refers to combinations in which all alleles are heterozygous or combinations in which a particular allele in the plurality of alleles is homozygous. In addition, when two or more alleles to be examined are not linked, the occurrence probability is always identical for each combination. However, if each allele is considered as a haplotype, the occurrence frequency of each allele combination would vary, and thus the occurrence probability would also vary. The occurrence probability can be calculated by basic calculation of combination probability.

More specifically, when three sets of alleles (A/a, A/a, and A/a) are examined, there are eight possible combinations of alleles. A number of combinations obtained by choosing two of the eight with allowing repeating (a number of combinations of diplotype) is 36. In such case, the following four combinations are those of diplotype in which all alleles are heterozygous, provided that each combination is expressed as “(paternal allele combination, maternal allele combination):” (AAA, aaa); (aAA, Aaa); (AaA, aAa); and (aaA, AAa). An occurrence probability can be calculated for each combination. In addition, examples of diplotype combinations in which a specific allele pair is homozygous include the following two combinations: (AAA, Aaa) and (AaA, AAa). Again, a occurrence probability can be calculated for each combination. Determination of the presence of chromosomal abnormalities can be made at an occurrence probability of 50% or higher.

Subsequently, a second determination function is used to determine a result as “positive,” “negative,” or “undetermined” with the use of database 3 of, the reliability of quantitative allelic changes in polymorphism regions and the threshold for determination. More specifically, database 3 is used to determine whether the quantitative allelic changes are highly reliable or less reliable. When the change is determined as less reliable, such results are designated as “negative.” When the change is determined as highly reliable, such results are designated as “positive.” At such time, values close to the threshold for determining the reliability level may be set as a range of undetermined results. Herein, the expression “quantitative allelic changes are less reliable” indicates that observed changes are derived from, for example, measurement variation and thus do not relate to chromosomal abnormalities. Also, the expression “quantitative allelic changes are highly reliable” indicates that observed changes do not fall within the range of measurement variation and thus chromosomal abnormalities are present. The reliability levels can be statistically calculated. The reliability of the threshold for determination can be freely set on a screen by users. The reliability to be used is preferably 95% and more preferably 99%. It is not absolutely necessary to use the aforementioned first determination function and second determination function in such order. The second function may be used before the first function. Alternatively, both functions may be simultaneously used.

Next, the presence or absence of chromosomal abnormalities is determined based on the results obtained by the first determination function and the results obtained by the second determination function. For determination, the reliability calculated by the first determination function and the reliability calculated by the second determination function are compared with each other. Then, more reliable determination results can be selected. Also, a test sample that has been determined to be “undetermined” by the second determination function can be re-determined by applying the results obtained by using the first determination function.

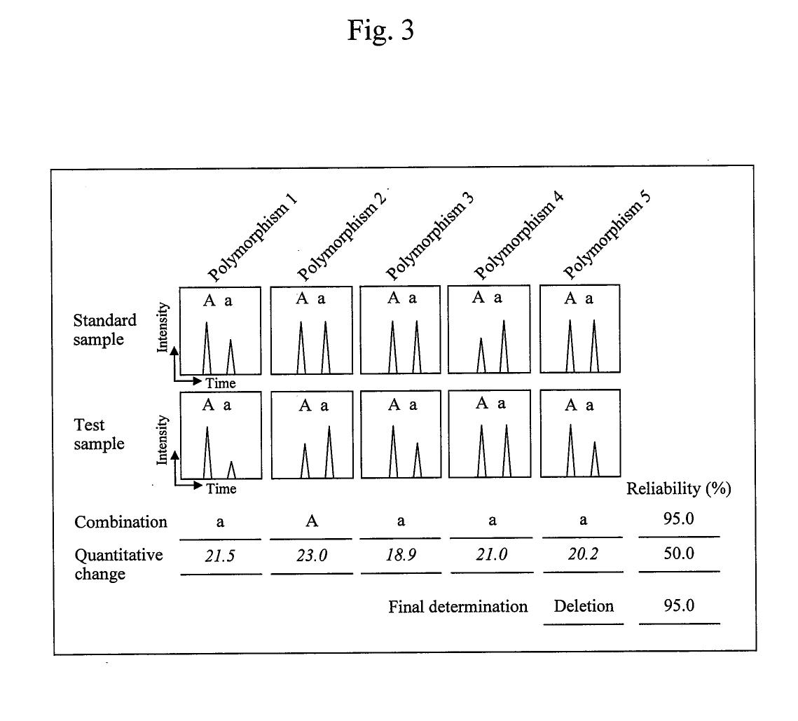

FIG. 3 shows an example of a display screen image upon the final determination of the present invention. As shown in the figure, an examination can be efficiently carried out by allowing a screen to display final determination results and the determination reliabilities. Preferably, the output screen presents at least determination results concerning the presence or absence of chromosomal abnormalities, the reliabilities of the results, and changes in the quantitative ratio of alleles. More preferably, the screen presents determination results obtained by the first determination function with the reliabilities thereof and determination results obtained by the second determination function with the reliabilities thereof.

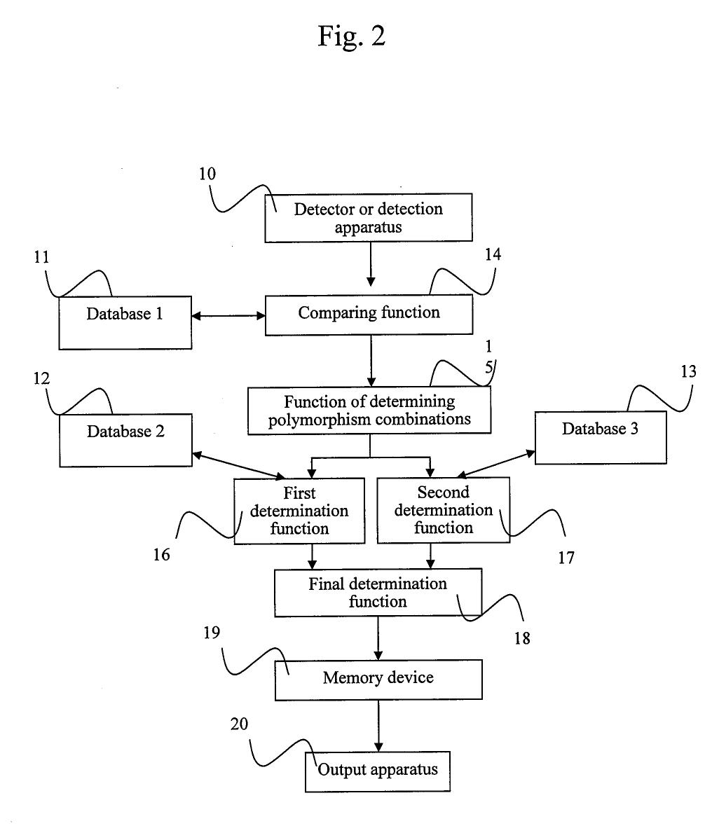

In the diagram of FIG. 2, the numeral 10 denotes a detector or apparatus for measuring the quantitative allele ratio for a standard sample or a test sample. The numeral 11 denotes a database of standard data regarding the quantitative allele ratio for each polymorphism in healthy individuals. The numeral 12 denotes a database of the statistically calculated reliabilities of quantitative changes and the thresholds that are predetermined based on the reliabilities provided that significant changes are obtained. The numeral 13 denotes a database of all polymorphism combinations to be tested and the occurrence frequencies thereof. The numeral 14 denotes a function of comparing a standard sample or standard data and measured data for a test sample and calculating quantitative allelic changes in a test sample. The numeral 15 denotes a function of comparing a standard sample or standard data and measured data for a test sample to determine a polymorphism type in each polymorphism region, and determine allele combinations in a test sample. The numeral 16 denotes a function of determining whether a result is “positive,” “negative,” or “undetermined” for quantitative allelic changes in each polymorphism region with the use of database of the reliabilities of the quantitative changes and the thresholds for determination. The numeral 17 denotes a function of determining whether a result is “positive,” “negative,” or “undetermined” based on the occurrence frequency of the combination of alleles in a test sample, by comparing a combination of alleles in a test sample with a database of all polymorphism combinations to be tested and the occurrence frequencies thereof, and storing the results or displaying the results on a screen. The numeral 18 denotes a determination function of comparing the determination results concerning quantitative changes obtained by the first determination function 16 with the determination results concerning polymorphism combinations obtained by the second determination function 17 and designating highly reliable results as final determination results. The numeral 19 denotes an apparatus for storing measurement data and determination results. The numeral 20 denotes an apparatus comprising a device for outputting the determination results.

The apparatus of the present invention first captures measurement data for a test sample and a standard sample from the detector or apparatus 10 for measuring the quantitative ratios of alleles in a standard sample and a test sample. Next, the measurement data for a test sample are compared with the measurement data for a standard sample or database 11 regarding the quantitative ratio of alleles in each polymorphism site in healthy individuals. Then, the function 14 is used to calculate quantitative allelic changes in a test sample and the function 15 is used to determine the polymorphism type in each polymorphism region so as to determine the combination of alleles in the test sample. The obtained results are stored in the memory device 19. The function 16 is used to determine whether the obtained result is “positive,” “negative,” or “undetermined” for quantitative allelic changes in each polymorphism region with the use of the database 12 of the reliabilities of quantitative changes and the predetermined thresholds. In parallel, the function 17 is used to compare the combination of alleles in a test sample with the database 13 regarding all polymorphism combinations to be tested and the occurrence frequencies thereof to determine whether the obtained result is “positive” or “negative” for the occurrence frequency of the combination of alleles in the test sample, and store the results or display the results on a screen. The function 18 is used to compare the determination results obtained by the function 16 with the determination results obtained by the function 17 in terms of reliability to select more reliable determination results, and determine whether the result is “positive” or “negative” as a final determination. Then, the determination result concerning quantitative changes obtained by the function 18, the determination reliability, the reliability of determination for the combination, and the reliability of the final determination are output to the output apparatus 20.

### EXAMPLES

The present invention is hereafter described in greater detail with reference to the following examples, although the scope of the present invention is not limited thereto.

### Example 1

### The Determination Method Using Combinations of Three Alleles in p53 Gene Region

### 1. Preparation of Test Samples

In this Example, a sample containing genomic nucleic acids extracted from peripheral blood was used as a standard sample and samples containing genomic nucleic acids extracted from bladder cancer tissues and urine of bladder cancer patients were used as test samples. Nucleic acids were extracted from cryopreserved samples containing genomic nucleic acids extracted from the peripheral blood, tissue, or urine by the method of Davis et al. (Basic Method in Molecular Biology, Elsevir Science Publishing) or the method of Sugano et al. (Lab. Invest. 68 pp. 361-366, Sugano et al. (1993)), wherein genomic nucleic acids are digested with proteinase K and extracted with phenol/chloroform. Briefly, samples were treated at 65° C. for 15 minutes. Tris-hydrochloric acid buffer (10 mmol/L) containing proteinase K (1 mg/mL), EDTA (10 mmol/L), and NaCl (150 mmol/L) was added thereto, followed by overnight incubation at 37° C. The obtained solution was added with a phenol/chloroform solution (phenol: chloroform=1:1 solution) in an equal volume and mixed, followed by centrifugation to extract nucleic acid. A 0.1-volume sodium acetate solution (3 mol/L) and 2.5-volume cold anhydrous ethanol were added to the extract, followed by cooling at −20° C. for 2 hours to precipitate nucleic acid. Glycogen (1 μg) was added as a carrier for ethanol precipitation to urine and cancer tissue samples to improve the nucleic acid recovery efficiency. The resulted solution was centrifuged to collect the precipitate, which was then washed with the addition of 80% ethanol (1 mL), followed by drying with a vacuum centrifugal concentrator. The resulted precipitate containing nucleic acids was redissolved in TE buffer. Collected peripheral blood was stored at 4° C. immediately after collection. Two days thereafter, nucleic acids were extracted therefrom. The extracted nucleic acids were cryopreserved at 25° C.

### 2. Detection of Alleles in Test Samples

In this Example, quantitative allelic changes were detected by the PCR-SSCP method. In the process of the detection, at first, an allele corresponding to a polymorphism site to be tested in a test sample was amplified by PCR. The amplified nucleic acid fragments were denatured into single strands. The allele quantity was detected as signal intensity by the SSCP method.

More specifically, PCR amplification conditions were set as listed in Table 1. Forward and reverse PCR primers listed in Table 2 were used as PCR primers for amplification and the 5′-end of either one of the primers was labeled with a FAM fluorescent dye.

TABLE 1
PCR amplification conditions

| Step | Temp (° C.) | Time (min) |
|---|---|---|
| 1 | 95 | 5:00 |
| 2 | 95 | 0:30 |
| 3 | 57 | 0:30 |
| 4 | 72 | 0:30 |
| 5 | 72 | 7:00 |
| 6 | 4 | for ever |

Step 2-Step 4: 30 cycles

TABLE 2
Primer sequence

| | | 5′-end | 3′-end | | Sequence |
|---|---|---|---|---|---|
| Sequence name | Direction | modification | modification | 5′-sequence-3′ | listing |
| p53 Exon4 | Reverse | None | None | CTGGGAAGGGACAG | SEQ ID |
| | strand | | | AAGATG | NO: 1 |
| | Forward | FAM | None | AGCTCCCAGAATGC | SEQ ID |
| | strand | | | CAGAG | NO: 2 |
| p53 Intron1 | Reverse | None | None | ACTGGCGCTGTGTG | SEQ ID |
| | strand | | | TAAATG | NO: 3 |
| | Forward | FAM | None | TCTTAGCTCGCGGT | SEQ ID |
| | strand | | | TGTTTC | NO: 4 |
| p53 Intron7 | Reverse | None | None | GTGATGAGAGGTGG | SEQ ID |
| | strand | | | ATGGGT | NO: 5 |
| | Forward | FAM | None | AGGTCAGGAGCCAC | SEQ ID |
| | strand | | | TTGCC | NO: 6 |

Genomic nucleic acids (templates) (0.1 μg) extracted from biological samples, primers (1.0 μM each), nucleotide triphosphates (dNTPs) (10 nM each), Tris-HCl buffer (pH 8.3) (10 μM), KCl (50 mM), MgCl₂ (1.5 mM), gelatin (0.001% (w/v)), and Taq nucleic acid polymerase (Perkin Elmer) (0.75 units) were mixed in a total volume of 30 μl. The obtained solution was subjected to a PCR reaction under the amplification conditions listed in Table 1. After the PCR reaction, the resultant was placed on ice (4° C.). Then, a PCR reaction solution (5 μl) obtained by amplifying standard nucleic acids and a PCR reaction solution (5 μl) obtained by amplifying test nucleic acids were mixed with a Voltex mixer to obtain a PCR reaction solution mixture.

Regarding preparation of a sample for electrophoresis used in the SSCP method, the order of addition of a reagent and a blunt-ended sample and the volumes thereof are given below. However, the order of addition thereof, the volumes thereof, and the denaturation conditions are not limited to the example given herein, as long as nucleic acid fragments can be denatured. Specifically, formamide as a DNA denaturant (39 μl) and an undiluted solution of a DNA amplification product (1.0 μl) were added to a microtube for analysis to obtain a total volume of 40 μl, followed by heat denaturation at 92° C. for 2 minutes and rapid cooling on ice (4° C.) for 5 minutes.

Each sample prepared for SSCP was subjected to SSCP electrophoresis with the use of a genetic analyzer 3100. Electrophoresis was conducted under conditions involving the use of Tris-HCl/glycine as an electrophoresis buffer and 15% GeneScan polymer as a separation polymer, the application of a voltage of 20 kV for 5 seconds for sample introduction, and the application of a voltage of 15 kV for 70 minutes for migration.

### 3. Measurement of Quantitative Allelic Changes

In this Example, signals (peak heights) from two alleles (NA, Na) in a standard sample were compared with signals from two alleles (TA, Ta) in a test sample. Quantitative allelic changes in a test sample were estimated by the equation described below (Genes. Chromosomes & Cancer 15 pp. 157-164 Sugano et al. (1996)). The letter “N” denotes a standard sample and the letter “T” denotes a test sample. The uppercase letter “A” denotes an allele of the two alleles which appears first in the electrophoresis and the lowercase letter “a” denotes the other allele observed which appears later.

Quantitative allelic change(%)=(NA/Na−TA/Ta)×100/{NA/Na}

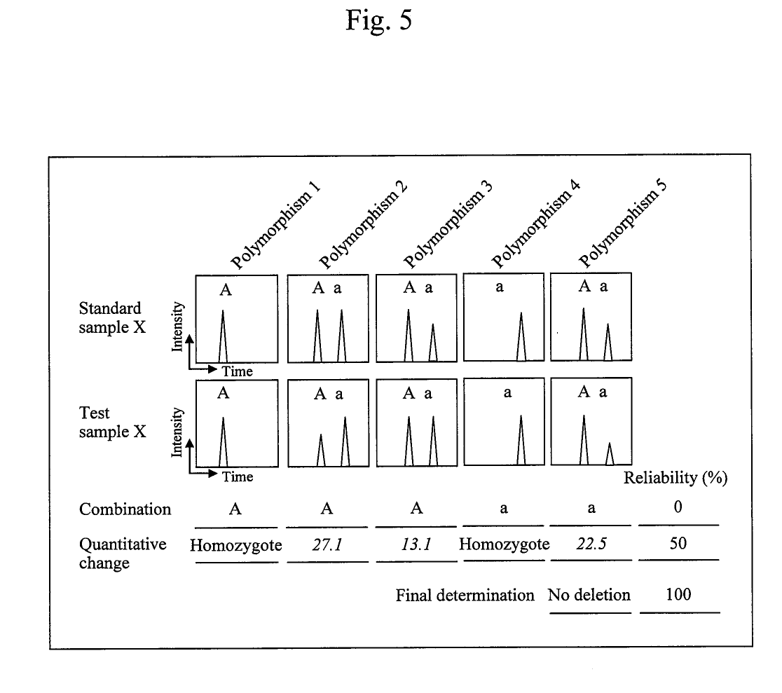

The above equation is used in a calculation wherein the quantity of an allele “A” decreases in a human having heterozygous alleles “A” and “a.” The letter “T” denotes a signal peak height from a test sample and the letter “N” denotes a signal peak height from a standard sample or obtained from standard data. Table 3 shows results obtained by measuring quantitative allelic changes in 16 test samples (1 to 16), each of which three different alleles were heterozygous. In this Example, the cases are shown in which all alleles were heterozygous. However, the present invention is not limited to this Example as long as at least two and more, preferably at least three, alleles among a plurality of examined alleles are heterozygous. FIG. 5 shows an example in which two of five alleles examined were homozygous.

TABLE 3
Results of measurement of quantitative
allelic changes in test samples

| | Cut off value | | |
|---|---|---|---|
| | 12 | 12 | 12 |
| Sample no. | p53 Exon4 | p53 Intron7 | p53 Intron1 |
| Test sample 1 | 22.5 | 13.8 | 21.9 |
| Test sample 2 | 2.6 | 3.1 | 3.2 |
| Test sample 3 | 1.9 | 10.8 | 29.8 |
| Test sample 4 | 16.3 | 14.6 | 28.8 |
| Test sample 5 | 3.5 | 25.8 | 24.9 |
| Test sample 6 | 0.6 | 4.5 | 2.0 |
| Test sample 7 | 15.2 | 13 | 16.2 |
| Test sample 8 | 72.8 | 71.2 | 72.3 |
| Test sample 9 | 4.4 | 4.7 | 3.9 |
| Test sample 10 | 6.1 | 10.1 | 14.2 |
| Test sample 11 | 36 | 35.3 | 38.4 |
| Test sample 12 | 1.0 | 0.9 | 3.9 |
| Test sample 13 | 9.0 | 1.2 | 3.6 |
| Test sample 14 | 1.2 | 6.0 | 10.9 |
| Test sample 15 | 92.9 | 89.3 | 92.5 |
| Test sample 16 | 16.8 | 2.0 | 5.7 |

### 4. Determination of Allele Combinations

In this Example, signals (peak heights) from two alleles (NA, Na) in a standard sample were compared with signals from two alleles (TA, Ta) in a test sample. Signals that decreased in the relevant polymorphism sites were detected. Allele combinations were determined based on the signal detection times. The obtained results were combined with the results listed in Table 3 and shown in Table 4. In this Example, alleles showing quantitative decreases were detected. However, it is also possible to detect alleles that do not exhibit any quantitative change.

TABLE 4
Results of detection of allele combination for test samples

| | Cut off value | | |
|---|---|---|---|
| | 12 | 12 | 12 |
| Sample no. | p53 Exon4 | p53 Intron7 | p53 Intron1 |
| Test sample 1 | a | a | A |
| Test sample 2 | A | A | A |
| Test sample 3 | A | A | A |
| Test sample 4 | a | A | A |
| Test sample 5 | a | A | A |
| Test sample 6 | A | a | A |
| Test sample 7 | A | A | A |
| Test sample 8 | A | A | a |
| Test sample 9 | a | a | a |
| Test sample 10 | A | a | A |
| Test sample 11 | A | A | a |
| Test sample 12 | a | A | a |
| Test sample 13 | a | A | A |
| Test sample 14 | a | A | A |
| Test sample 15 | A | A | a |
| Test sample 16 | a | A | a |

### 5. Determination of the Presence or Absence of Chromosomal Abnormalities Based on Quantitative Allelic Changes

In this Example, Table 5 shows quantitative allelic changes for determination of the presence or absence of abnormalities. Quantitative allelic changes of 40% or more were designated as “positive” (written in bold). Quantitative allelic changes of 7% or more and less than 40% were designated as “undetermined” (written in Italic). Quantitative allelic changes of less than 7% were designated as “negative” (written in normal font). In addition, for comparison with the method of the present invention, determination results of quantitative changes obtained by a conventional determination method are also shown. In the conventional method, determinations were carried out using the cut-off value based on 3SD of measurement variation when the DNA concentration is sufficiently high.

In this Example, measurement variation larger than usual measurement variation were frequently observed. This resulted from increases in variation due to low DNA concentrations as shown in the conventional method (Laboratory Investigation (2004) 84, 649-657). Such increases in variation due to DNA concentrations would vary depending on DNA concentrations. In such case, variation would vary depending on test sample DNA concentrations. Therefore, in this Example, the determination method of the present invention is described wherein quantitative changes of 40% or less are always considered as with low reliability.

TABLE 5
Determination of the presence or absence of chromosomal
abnormalities based on quantitative allelic changes in test samples

| The method of the present invention | | | | The conventional determination method | | | |
|---|---|---|---|---|---|---|---|
| | Cut off value | | | | Cut off value | | |
| | 12 | 12 | 12 | | 10 | 10 | 10 |
| | p53 | p53 | p53 | | p53 | p53 | p53 |
| Sample no. | Exon4 | Intron7 | Intron1 | Sample no. | Exon4 | Intron7 | Intron1 |
| Combination | a | a | A | Combination | a | a | A |
| Test sample 1 | 22.5 | 13.8 | 21.9 | Test sample 1 | 22.5 | 13.8 | 21.9 |
| Combination | A | A | A | Combination | A | A | A |
| Test sample 2 | 2.6 | 3.1 | 3.2 | Test sample 2 | 2.6 | 3.1 | 3.2 |
| Combination | A | A | A | Combination | A | A | A |
| Test sample 3 | 1.9 | 10.8 | 29.8 | Test sample 3 | 1.9 | 10.8 | 29.8 |
| Combination | a | A | A | Combination | a | A | A |
| Test sample 4 | 16.3 | 14.6 | 28.8 | Test sample 4 | 16.3 | 14.6 | 28.8 |
| Combination | a | A | A | Combination | a | A | A |
| Test sample 5 | 3.5 | 25.8 | 24.9 | Test sample 5 | 3.5 | 25.8 | 24.9 |
| Combination | A | a | A | Combination | A | a | A |
| Test sample 6 | 0.6 | 4.5 | 2.0 | Test sample 6 | 0.6 | 4.5 | 2.0 |
| Combination | A | A | A | Combination | A | A | A |
| Test sample 7 | 15.2 | 13 | 16.2 | Test sample 7 | 15.2 | 13 | 16.2 |
| Combination | A | A | a | Combination | A | A | a |
| Test sample 8 | 72.8 | 71.2 | 72.3 | Test sample 8 | 72.8 | 71.2 | 72.3 |
| Combination | a | a | a | Combination | a | a | a |
| Test sample 9 | 4.4 | 4.7 | 3.9 | Test sample 9 | 4.4 | 4.7 | 3.9 |
| Combination | A | a | A | Combination | A | a | A |
| Test sample 10 | 6.1 | 10.1 | 14.2 | Test sample 10 | 6.1 | 10.1 | 14.2 |
| Combination | A | A | a | Combination | A | A | a |
| Test sample 11 | 36 | 35.3 | 38.4 | Test sample 11 | 36 | 35.3 | 38.4 |
| Combination | a | A | a | Combination | a | A | a |
| Test sample 12 | 1.0 | 0.9 | 3.9 | Test sample 12 | 1.0 | 0.9 | 3.9 |
| Combination | a | A | A | Combination | a | A | A |
| Test sample 13 | 9.0 | 1.2 | 3.6 | Test sample 13 | 9.0 | 1.2 | 3.6 |
| Combination | a | A | A | Combination | a | A | A |
| Test sample 14 | 1.2 | 6.0 | 10.9 | Test sample 14 | 1.2 | 6.0 | 10.9 |
| Combination | A | A | a | Combination | A | A | a |
| Test sample 15 | 92.9 | 89.3 | 92.5 | Test sample 15 | 92.9 | 89.3 | 92.5 |
| Combination | a | A | a | Combination | a | A | a |
| Test sample 16 | 16.8 | 2.0 | 5.7 | Test sample 16 | 16.8 | 2.0 | 5.7 |

### 6. Determination of the Presence or Absence of Chromosomal Abnormalities Based on Allele Combination Frequencies

Next, Table 6 shows all possible combinations of three alleles in the p53 gene region and the occurrence frequency of each combination obtained from haplotype analysis results of approximately 100 individuals. Table 7 shows results obtained by calculating the occurrence probabilities of combinations in which all alleles are heterozygous based on the above occurrence frequencies. In this Example, an occurrence probability is considered as a reliability of a combination.

TABLE 6
All possible combinations of three alleles in
the p53 gene region and occurrence frequencies

| | | | | Occurrence |
|---|---|---|---|---|
| | p53 | p53 | p53 | frequency |
| | Exon 4 | Intron 7 | Intron 1 | (%) |
| Combination 1 | a | a | A | 71.8 |
| Combination 2 | A | A | a | 23.9 |
| Combination 3 | A | a | A | 3.8 |
| Combination 4 | A | A | A | 0.4 |
| Combination 5 | a | a | a | 0.1 |
| Combination 6 | a | A | A | 0.0 |
| Combination 7 | a | A | a | 0.0 |
| Combination 8 | A | a | a | 0.0 |

TABLE 7
Combinations of alleles in which all alleles
are heterozygous and occurrence probabilities

| | | | | Occurrence |
|---|---|---|---|---|
| | p53 | p53 | p53 | probability |
| Combination | Exon 4 | Intron 7 | Intron 1 | (%) |
| Combination 1 | a | a | A | 99.7 |
| Combination 2 | A | A | a | |
| | Hetero | Hetero | Hetero | |
| Combination 4 | A | A | A | 0.3 |
| Combination 5 | a | a | a | |
| | Hetero | Hetero | Hetero | |
| Combination 3 | A | a | A | 0.0 |
| Combination 7 | a | A | a | |
| | Hetero | Hetero | Hetero | |
| Combination 6 | a | A | A | 0.0 |
| Combination 8 | A | a | a | |
| | Hetero | Hetero | Hetero | |

As seen from Table 7, undetermined test values were designated as “positive” or “negative” in the following manner. When an allele combinations 1 and 2, an occurrence probability of which is 99.7%, was observed as a deletion pattern, such a test value was designated as “positive.” When a different combination was observed as a deletion pattern, such a test value was designated as “negative.” Regarding the determination results of the quantitative changes obtained by the methods shown in Table 5, undetermined test results were determined by the above method. Results designated as “positive” are written in bold in Table 8.

TABLE 8
Determination results for undetermined test values obtained by the
determination method of the present invention

| The method of the present invention | | | | The conventional determination method | | | |
|---|---|---|---|---|---|---|---|
| | Cut off value | | | | Cut off value | | |
| | 7-40 | 7-40 | 7-40 | | 10 | 10 | 10 |
| | p53 | p53 | p53 | | p53 | p53 | p53 |
| Sample no. | Exon4 | Intron7 | Intron1 | Sample no. | Exon4 | Intron7 | Intron1 |
| Combination | a | a | A | Combination | a | a | A |
| Test sample 1 | 22.5 | 13.8 | 21.9 | Test sample 1 | 22.5 | 13.8 | 21.9 |
| Combination | A | A | A | Combination | A | A | A |
| Test sample 2 | 2.6 | 3.1 | 3.2 | Test sample 2 | 2.6 | 3.1 | 3.2 |
| Combination | A | A | A | Combination | A | A | A |
| Test sample 3 | 1.9 | 10.8 | 29.8 | Test sample 3 | 1.9 | 10.8 | 29.8 |
| Combination | a | A | A | Combination | a | A | A |
| Test sample 4 | 16.3 | 14.6 | 28.8 | Test sample 4 | 16.3 | 14.6 | 28.8 |
| Combination | a | A | A | Combination | a | A | A |
| Test sample 5 | 3.5 | 25.8 | 24.9 | Test sample 5 | 3.5 | 25.8 | 24.9 |
| Combination | A | a | A | Combination | A | a | A |
| Test sample 6 | 0.6 | 4.5 | 2.0 | Test sample6 | 0.6 | 4.5 | 2.0 |
| Combination | A | A | A | Combination | A | A | A |
| Test sample 7 | 15.2 | 13 | 16.2 | Test sample 7 | 15.2 | 13 | 16.2 |
| Combination | A | A | a | Combination | A | A | a |
| Test sample 8 | 72.8 | 71.2 | 72.3 | Test sample 8 | 72.8 | 71.2 | 72.3 |
| Combination | a | a | a | Combination | a | a | a |
| Test sample 9 | 4.4 | 4.7 | 3.9 | Test sample 9 | 4.4 | 4.7 | 3.9 |
| Combination | A | a | A | Combination | A | a | A |
| Test sample 10 | 6.1 | 10.1 | 14.2 | Test sample10 | 6.1 | 10.1 | 14.2 |
| Combination | A | A | a | Combination | A | A | a |
| Test sample 11 | 36 | 35.3 | 38.4 | Test sample 11 | 36 | 35.3 | 38.4 |
| Combination | a | A | a | Combination | a | A | a |
| Test sample 12 | 1.0 | 0.9 | 3.9 | Test sample 12 | 1.0 | 0.9 | 3.9 |
| Combination | a | A | A | Combination | a | A | A |
| Test sample 13 | 9.0 | 1.2 | 3.6 | Test sample 13 | 9.0 | 1.2 | 3.6 |
| Combination | a | A | A | Combination | a | A | A |
| Test sample 14 | 1.2 | 6.0 | 10.9 | Test sample 14 | 1.2 | 6.0 | 10.9 |
| Combination | A | A | a | Combination | A | A | a |
| Test sample 15 | 92.9 | 89.3 | 92.5 | Test sample 15 | 92.9 | 89.3 | 92.5 |
| Combination | a | A | a | Combination | a | A | a |
| Test sample 16 | 16.8 | 2.0 | 5.7 | Test sample 16 | 16.8 | 2.0 | 5.7 |

### 7. Final Determination of the Presence or Absence of Chromosomal Abnormalities

Based on the results shown in Table 8, the comprehensive determination results obtained from the three alleles were shown as final determination results in Table 9 each of which designated as “positive” or “negative”. In the conventional determination method, in cases in which two or more out of three results are “positive” are finally designated as “positive.” Further, in order to evaluate whether the determination results are true, a plurality of other polymorphism sites were examined to show the actual presence or absence of abnormalities. The results were considered as true determinations and shown in Table 9. Based on the true determination results, the sensitivity (%) and the specificity (%) were calculated for the determination method of the present invention and for the conventional method. In addition, when the determination result obtained by the method of the present invention was designated as “positive,” the reliability of the determination was calculated. Further, the reliability was calculated for a result designated as “positive” and for a result designated as “negative.” In the reliability calculation method used in this Example, the reliability was determined to be 100% when a quantitative allelic change is not less than 40% or less than 7%. The occurrence probability was regarded as the reliability when the quantitative allelic change fell within the range of undetermined results.

TABLE 9
Final determination results

| | The | | The | |
|---|---|---|---|---|
| | present | Reliability | conventional | |
| | method | (%) | method | True result |
| Test sample 1 | Positive | 87.2 | Positive | Positive |
| Test sample 2 | Negative | — | Negative | Negative |
| Test sample 3 | Negative | — | Positive | Negative |
| Test sample 4 | Negative | — | Positive | Negative |
| Test sample 5 | Negative | — | Positive | Negative |
| Test sample 6 | Negative | — | Negative | Negative |
| Test sample 7 | Negative | — | Positive | Negative |
| Test sample 8 | Positive | 100 | Positive | Positive |
| Test sample 9 | Negative | — | Negative | Negative |
| Test sample 10 | Negative | — | Positive | Negative |
| Test sample 11 | Positive | 100 | Positive | Positive |
| Test sample 12 | Negative | — | Negative | Negative |
| Test sample 13 | Negative | — | Negative | Negative |
| Test sample 14 | Negative | — | Negative | Negative |
| Test sample 15 | Positive | 100 | Positive | Positive |
| Test sample 16 | Negative | — | Negative | Negative |
| Sensitivity (%) | 100 | — | 100 | — |
| Specificity (%) | 100 | — | 41.6 | — |

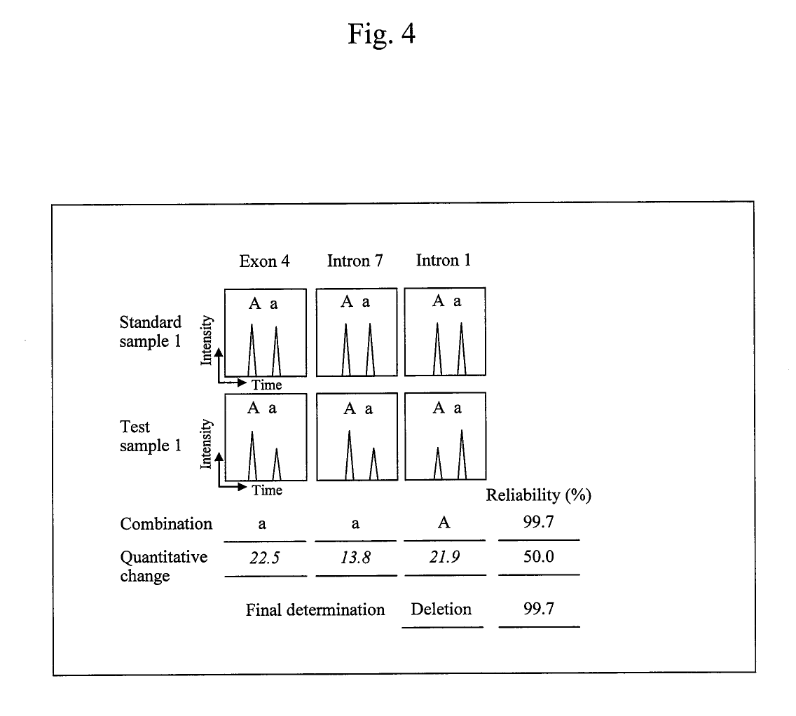

Consequently, the sensitivity, at which the “positive” result can be accurately determined, was the same for the both methods. However, in the method of the present invention, the specificity, at which the “negative” result can be accurately determined, was improved by 50% or more so that the determination results were completely identical to those in the true results. In this Example, Test samples 8 and 15, in which the allelic changes are as high as 70% or more, have indeed the combination of the alleles with a high occurrence frequency. This indicates the validity of the present invention. In addition, it is considered that similar changes are found in closely located alleles in an identical sample. Thus, it is probably possible to attain a false-positive result based on differences in quantitative changes in various alleles. However, as in the cases of Test samples 4 and 7, even when three alleles showed similar changes, it was possible to accurately designate the obtained result as “negative” based on the allele combination. Further, FIG. 4 shows an output display image of the final determination results for Test sample 1 in this Example.

All publications, patents, and patent applications cited herein are incorporated herein by reference in their entirety.

### INDUSTRIAL APPLICABILITY

The present invention can be used for chromosome tests such as LOH assays and CGH tests.

### Free Text of Sequence Listings

### SEQ ID NO: 1: Primer

### SEQ ID NO: 2: Primer

### SEQ ID NO: 3: Primer

### SEQ ID NO: 4: Primer

### SEQ ID NO: 5: Primer

SEQ ID NO: 6: Primer

1. A method of determining chromosomal abnormalities in a test sample by examining quantitative ratios of alleles in genetic polymorphism sites, comprising the steps of:
measuring quantitative changes in alleles in two or more linked polymorphism sites to identify alleles each of which exhibits quantitative changes when compared with those in a standard sample; and
determining the presence or absence of chromosomal abnormalities based on the occurrence frequency of a combination of the alleles when the allele combination is considered as a haplotype.

2. The method according to claim 1, wherein the determination based on the occurrence frequency is carried out with the use of a test sample that is difficult to determine in terms of chromosomal abnormalities based on quantitative changes in the alleles since the changes are within an error range in the sample.

3. The method according to claim 1, further comprising the following steps:
1) calculating a reliability of quantitative changes in alleles exhibiting changes in gene quantity when compared with those in a standard sample;
2) determining the presence or absence of chromosomal abnormalities based on quantitative changes in the alleles and the occurrence frequency of a combination of the alleles; and
3) comparing the occurrence frequency of the combination of the alleles and the reliability of the quantitative changes in the alleles to designate the higher result as a final determination result.

4. The method according to claim 1, wherein the test sample is determined as having chromosomal abnormalities when the occurrence frequency of the combination of the alleles is 1% or more, given that the allele combination is considered as a haplotype.

5. The method according to claim 1, wherein the test sample is determined as having chromosomal abnormalities when the test sample comprises a diplotype with an occurrence frequency of 50% or more, given that the allele combination is considered as a haplotype.

6. A program for determining chromosomal abnormalities by examining quantitative ratios of alleles in genetic polymorphism sites, comprising:
a means of inputting quantitative changes in the alleles in two or more linked polymorphism sites in a test sample in comparison with a standard sample;
a means of identifying alleles each of which exhibits quantitative changes when compared with those in a standard sample; and
a means of determining the presence or absence of chromosomal abnormalities based on the occurrence frequency of a combination of the alleles, when the allele combination is considered as a haplotype.

7. The program according to claim 6, further comprising the following means:
1) a means of calculating a reliability of quantitative changes in alleles exhibiting quantitative changes when compared with those in a standard sample;
2) a means of determining the presence or absence of chromosomal abnormalities based on quantitative changes in the alleles and the occurrence frequency of a combination of the alleles; and
3) a means of comparing the occurrence frequency of the combination of the alleles and the reliability of quantitative changes in the alleles, determining the higher result as a final determination result, and outputting the result.

8. The program according to claim 7, further comprising a means of outputting the reliability of the final determination result.

9. The program according to claim 7, further comprising a means of outputting the combination of the alleles, the occurrence frequency thereof, the reliability of data on quantitative changes in the alleles, and the reliability of the final determination result.

10. The program according to claim 6, further comprising a means of storing various forms of data derived from a test sample, including the occurrence frequencies of combinations obtained by examination, and the occurrence frequencies of individual haplotypes obtained by re-calculation with the addition of the data.

11. The program according to claim 6, wherein the test sample is determined as having chromosomal abnormalities when the occurrence frequency of the combination of the alleles is 1% or more, given that the allele combination is considered as a haplotype.

12. The program according to claim 6, wherein the test sample is determined as having chromosomal abnormalities when the test sample comprises a diplotype with an occurrence frequency of 50% or more, given that the allele combination is considered as a haplotype.

13. An apparatus for determining chromosomal abnormalities by examining quantitative ratios of alleles in genetic polymorphism sites, comprising:
a means of determining quantitative changes in alleles in two or more linked polymorphism sites in a test sample in comparison with a standard sample;
a means of identifying alleles each of which exhibits quantitative changes when compared with those in a standard sample; and
a means of determining the presence or absence of chromosomal abnormalities based on the occurrence frequency of a combination of the alleles, given that the allele combination is considered as a haplotype.

14. The apparatus according to claim 13, further comprising the following means:
1) a means of calculating a reliability of quantitative changes in alleles exhibiting quantitative changes when compared with those in a standard sample;
2) a means of determining the presence or absence of chromosomal abnormalities based on quantitative changes in the alleles and the occurrence frequency of a combination of the alleles; and
3) a means of comparing the occurrence frequency of the combination of the alleles and the reliability of data on quantitative changes in the alleles, determining the higher result as a final determination results, and presenting the result.

15. The apparatus according to claim 14, further comprising a means of presenting the reliability of the final determination result.

16. The apparatus according to claim 14, further comprising a means of presenting the combination of the alleles, the occurrence frequency thereof, the reliability of quantitative changes in the alleles, and the reliability of the final determination result.

17. The apparatus according to claim 14, further comprising a means of storing various forms of data from a test sample, including the occurrence frequencies of combinations obtained by examination, and the occurrence frequencies of individual haplotypes obtained by re-calculation with the addition of the data.

18. The apparatus according to claim 13, wherein the test sample is determined as having chromosomal abnormalities when the occurrence frequency of the combination of the alleles is 1% or more, given that the allele combination is considered as a haplotype.

19. The apparatus according to claim 13, wherein the test sample is determined as having chromosomal abnormalities when the test sample comprises a diplotype with an occurrence frequency of 50% or more, given that the allele combination is considered as a haplotype.


SEQUENCE LISTING (6 sequences)

SEQ ID NO: 1 - DNA, 20 bp, Artificial
  primer
ctgggaagggacagaagatg

SEQ ID NO: 2 - DNA, 19 bp, Artificial
  primer
agctcccagaatgccagag

SEQ ID NO: 3 - DNA, 20 bp, Artificial
  primer
actggcgctgtgtgtaaatg

SEQ ID NO: 4 - DNA, 20 bp, Artificial
  primer
tcttagctcgcggttgtttc

SEQ ID NO: 5 - DNA, 20 bp, Artificial
  primer
gtgatgagaggtggatgggt

SEQ ID NO: 6 - DNA, 19 bp, Artificial
  primer
aggtcaggagccacttgcc